(19) United States
(12) Patent Application Publication
(10) Pub. No.: US 2008/0167262 A1
(43) Pub. Date: Jul. 10, 2008

(54) METHODS AND COMPOSITIONS FOR DIAGNOSING AND TREATING PROSTATE CANCER

(75) Inventors: Kathleen A. Cooney, Ann Arbor, MI (US); Julie A. Douglas, Ann Arbor, MI (US); Albert M. Levin, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 11/937,580

(22) Filed: Nov. 9, 2007

Related U.S. Application Data
(60) Provisional application No. 60/857,948, filed on Nov. 10, 2006.

(51) Int. Cl. A61K 31/70; C12Q 1/68

(52) U.S. Cl. 514/44; 435/6

(57) ABSTRACT

The present invention relates to compositions and methods for cancer research, diagnosis, and treatment, including but not limited to, cancer markers. In particular, the present invention relates to BRCA1 markers for prostate cancer.

This application claims priority to provisional patent application 60/857,948, filed Nov. 10, 2006, which is herein incorporated by reference in its entirety.

This invention was made with government support under CA79596 and CA69568 from the National Institutes of Health. The government has certain rights in the invention.

### FIELD OF THE INVENTION

The present invention relates to compositions and methods for cancer research, diagnosis, and treatment, including but not limited to, cancer markers. In particular, the present invention relates to BRCA1 markers for prostate cancer.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]); Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to compositions and methods for cancer research, diagnosis, and treatment, including but not limited to, cancer markers. In particular, the present invention relates to BRCA1 markers for prostate cancer.

Accordingly, in some embodiments, the present invention provides a series of BRCA1 polymorphisms that are associated with prostate cancer. The BRCA1 alleles of the present invention find use in providing diagnostic and prognostic information. The alleles also find use as therapeutic targets and in research (e.g., drug screening) applications.

For example, in some embodiments, the present invention provides a method of diagnosing prostate cancer, comprising detecting the presence or absence of a BRCA1 single nucleotide polymorphism (e.g., rs1799950, rs3737559, or rs799923) in a sample (e.g., a biopsy, urine, or blood sample) from a subject; and determining the presence or absence of prostate cancer in the sample based on the presence or absence of the BRCA1 single nucleotide polymorphism. In some embodiments, the presence of the BRCA1 single nucleotide polymorphism in the sample is indicative of prostate cancer in the sample. In some embodiments, the single nucleotide polymorphism is rs1799950 (e.g., a Gln356Arg substitution in a BRCA1 polypeptide expressed from the rs1799950 single nucleotide polymorphism).

The present invention further provides a kit comprising reagents for detecting the presence or absence of a BRCA1 single nucleotide polymorphism (e.g., rs1799950, rs3737559, or rs799923).

The present invention additionally provides a method, comprising: contacting a prostate cancer cell expressing a BRCA1 gene comprising a BRCA1 single nucleotide polymorphism (e.g., rs1799950, rs3737559, or rs799923) with a test compound; and identifying a test compound that impairs proliferation of or kills the cell. In some embodiments, the method further comprises the step of administering the test compound that impairs proliferation of or kills the cell to a subject diagnosed with prostate cancer. In certain embodiments, the present invention further comprises the step of marketing the test compound as a drug for treating prostate cancer.

### DESCRIPTION OF THE FIGURES

FIG. 1 shows haplotype tag SNPs (htSNPs) and pair-wise linkage disequilibrium (LD) as measured by R² in a 200 kb-region around the BRCA1 gene.

FIG. 2 shows non-parametric multipoint linkage analysis for prostate cancer on chromosome 17.

### DEFINITIONS

To facilitate an understanding of the present invention, a number of terms and phrases are defined below:

As used herein, the term “haplotype” refers to a group of closely linked alleles that are inherited together.

As used herein, the term “haplotype clade” or “clade” refers to any group of haplotypes that are all more similar to one another than any of them is to any other haplotype. Clades may be identified, for example, by performing statistical cluster analysis. The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing prostate tissue in a subject” refers to the identification of one or more properties of a prostate tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient.

As used herein, the term “a reagent that specifically detects the presence or absence of a cancer marker” refers to reagents used to detect the expression of one or more cancer markers (e.g., including but not limited to, the cancer markers of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “initial diagnosis” refers to results of initial cancer diagnosis (e.g. the presence or absence of cancerous cells). An initial diagnosis does not include information about the stage of the cancer of the risk of prostate specific antigen failure.

As used herein, the term “biopsy tissue” refers to a sample of tissue (e.g., prostate tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length nRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i. e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “polymorphic locus” is a locus present in a population that shows variation between members of the population (e.g., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

As used herein, the term “hybridization” is used in reference to the pairing of complementary nucleic acids. Hybridization and the strength of hybridization (i. e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]. Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μ/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer research, diagnosis, and treatment, including but not limited to, cancer markers. In particular, the present invention relates to BRCA1 markers for prostate cancer. Accordingly, the present invention provides methods and kits for the detection of markers, as well as drug screening and therapeutic applications.

Experiments conducted during the course of development of the present invention identified three common SNPs (i.e., minor allele frequency greater than 5%) in the BRCA1 gene, including rs1799950, rs3737559, and rs799923, which are associated with prostate cancer and are in weak LD with each other. The strongest evidence for prostate cancer association was for SNP rs1799950, or equivalently Gln356Arg. It is estimated that men carrying at least one Arg356 allele are approximately 2.3 times more likely to develop prostate cancer than non-carriers. This SNP also explains, in part, previously reported linkage region on 17q in 172 GWS families (Lange et al., Prostate 57:326-334. 2003), which included 76 of the 338 families from a family-based association study. Thus, evidence of both linkage and association between prostate cancer and common variation in the BRCA1 gene were identified. The results also indicate that BRCA1 Gln356Arg accounts partially for the original evidence of linkage on chromosome 17q21, indicating that there are likely multiple functional variants in this region that influence prostate cancer susceptibility.

Significant evidence of linkage and association was found in several different strata, including the subset of families in which men were diagnosed with prostate cancer at <50 years of age (for SNP rs1799950) and the subset of families with 3 or more affected men (for SNPs rs2271539 and rs3737559). The latter is consistent with the linkage results on 17q, where the evidence for linkage was increased by analyzing only the subset of families with multiple affected men (Lange et al., 2003, supra). These findings suggest that particular subsets of families, possibly those with more heritable forms of prostate cancer, contributed disproportionately to the results. These strata defined only partially overlapping sets of families (i.e., less than 50% of informative families in one stratum were included in another stratum), suggesting the presence of genetic heterogeneity.

Several groups have previously examined the role of BRCA1 Gln356Arg in cancer susceptibility. For example, some (Dunning et al., Hum. Mol. Genet. 6:285-289. 1997; Soucek et al., Breast Cancer Res Treat. 2006) but not all groups (Menzel et al., Br. J Cancer 90:1989-1994. 2004; Cox et al., Breast Cancer Res 7:R171-R175. 2005; Freedman et al., Cancer Res 65:7516-7522. 2005) have reported an association between Gln356Arg and breast cancer in case-control studies, with the Arg356 allele conferring a reduction in breast cancer risk. At least three other groups (Janezic et al., Hum. Mol. Genet. 8:889-897. 1999; Wenham et al., Clin. Cancer Res 9:4396-4403. 2003; Auranen et al., Int. J Cancer 117:611-618. 2005) have also examined the role of BRCA1 Gln356Arg in case-control studies of ovarian cancer but found no evidence of an association.

BRCA1 Gln356Arg is located in a region of exon 11 that binds Rad50 (which is part of the DNA damage repair complex) and the transcriptional repressor ZBRK1. Some (Ng and Henikoff, Genome Res. 12:436-446. 2002; Burk-Herrick et al., Mamm. Genome 17:257-270. 2006) but not all (Fleming et al., Proc. Natl. Acad. Sci. U.S.A 100:1151-1156. 2003) computational tools predict that the Gln356Arg substitution adversely affects BRCA1 protein function. These tools rely on comparative evolutionary analyses to establish whether mutations occur in regions that are conserved and/or are evolving under selective pressure. For example, the Sorting Intolerant from Tolerant (SIFT) program (Ng and Henikoff, supra) uses sequence conservation information to predict whether an amino acid substitution will affect protein function. Although BRCA1 is located in a region showing relatively poor sequence conservation between species, Gln356Arg is predicted to be an “intolerant” substitution by SIFT. In addition, when analyzed in the context of natural selection information (i.e., purifying, neutral, or diversifying), Burk-Herrick et al. (supra) recently classified BRCA1 Gln356Arg as an “oncogenic” risk mutation. The present invention is not limited to a particular mechanism. Indeed, an understanding of the mechanism is not necessary to practice the present invention. Nonetheless, the mechanism by which the Arg356 allele of BRCA1 might increase the risk of prostate cancer is unclear, although the GlnArg356 substitution generates a run of three positively charged residues (LysArgLys), which could conceivably alter the properties of the protein.

Accordingly, in some embodiments, the present invention provides diagnostic and research methods for diagnosing prostate cancer based on the presence or absence of BRCA1 polymorphisms.

### I. BRCA1 Prostate Cancer Markers

As described above, the present invention provides BRCA1 alleles that are associated with prostate cancer (e.g., SNPs rs1799950, rs3737559, and rs799923). Such markers find use in the diagnosis and characterization of prostate cancer.

### A. Detection of BRCA1 Alleles

In some embodiments, the present invention provides methods for detection of BRCA1 alleles associated with prostate cancer. In some embodiments, alleles are detected in tissue samples (e.g., biopsy tissue). In other embodiments, alleles are detected in bodily fluids (e.g., including but not limited to, plasma, serum, whole blood, mucus, and urine). Exemplary methods for determining BRCA1 polymorphisms are described below.

1. Direct sequencing Assays

In some embodiments of the present invention, BRCA1 polymorphic sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, and automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Hybridization Assays

In preferred embodiments of the present invention, variant sequences are detected using a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.
a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.
b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.
c. Enzymatic Detection of Hybridization

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin). Numerous other assays are known in the art.

4. Other Detection Assays

Additional detection assays that are suitable for use in the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); INVADER assay, Third Wave Technologies; See e.g., U.S. Pat. Nos. 5,846,717, 6,090,543; 6,001,567; 5,985,557; and 5,994,069; each of which is herein incorporated by reference; cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

5. Mass Spectroscopy Assay

In some embodiments, a MassARRAY system (Sequenom, San Diego, Calif.) is used to detect variant sequences (See e.g., U.S. Pat. Nos. 6,043,031; 5,777,324; and 5,605,798; each of which is herein incorporated by reference). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

### B. Data Analysis

In some embodiments, a computer-based analysis program is used to translate the raw data generated by the detection assay (e.g., the presence, absence, or amount of a given BRCA1 allele) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present or the subtype of cancer) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### C. Kits

In some embodiments, the present invention provides kits for the detection of BRCA1 polymorphisms. In some embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results. In some embodiments, individual probes and reagents for detection of BRCA1 polymorphisms are provided as analyte specific reagents. In other embodiments, the kits are provided as in vitro diagnostics.

### II. Drug Screening

In some embodiments, the present invention provides drug screening assays (e.g., to screen for anticancer drugs). The screening methods of the present invention utilize cancer markers identified using the methods of the present invention (e.g., including but not limited to, BRCA1 alleles). For example, in some embodiments, the present invention provides methods of screening for anti-cancer (e.g., prostate cancer) compounds that are effective against the BRCA1 alleles of the present invention.

For example, in some embodiments, cells or organisms expressing BRCA1 alleles identified as being associated with prostate cancer are expressed in prostate cancer cell lines or normal cell lines and the effect of test compounds on the proliferation of the cells is assayed. In some embodiments, the test compounds are known anti-cancer compounds. In other embodiments, the test compounds are new compounds.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

### III. Transgenic Animals Expressing BRCA1 Alleles

The present invention contemplates the generation of transgenic animals comprising a BRCA1 allele (e.g., those identified herein as being associated with prostate cancer). In preferred embodiments, the transgenic animal displays an altered phenotype (e.g., increased incidence of prostate cancer) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

### EXAMPLE 1

### BRCA1 Alleles In Prostate Cancer

### A. Materials and Methods

### Study Subjects

The PCGP is a large, ongoing family-based study designed to map and clone genes predisposing to inherited forms of prostate cancer. Enrollment into the PCGP is restricted to (1) families with two or more living members with prostate cancer in a first-or second-degree relationship or (2) men diagnosed with prostate cancer at ≦55 years of age without a family history of the disease. All participants are asked to provide a blood sample, extended family history information, and access to medical records. For the present investigation, 338 families in which DNA was available from at least one pair of brothers discordant for prostate cancer were utilized. These discordant sibling pairs (DSPs) were selected from a single generation to mitigate potential cohort effects. The oldest available unaffected brother from each family was preferentially enrolled to maximize the probability that unaffected men were truly unaffected and not simply unaffected by virtue of being younger than their affected brother(s). Additional male siblings as well as multiple sibships from the same family were included if DNA was available.

The majority of the PCGP families were recruited directly from the University of Michigan Comprehensive Cancer Center. Other sources included direct patient or physician referrals. Diagnosis of prostate cancer was confirmed by review of pathology reports or medical records, and age at diagnosis was calculated from the date of the first biopsy positive for prostate cancer. Cases were classified as clinically aggressive if they met at least one of the following criteria: (1) pathologic Gleason sum >7, (2) pathologic stage T3b (pT3b) tumor (indicating seminal vesicle involvement) or pT4 or N1 (positive regional lymph nodes), (3) pathologic Gleason sum of 7 and a positive margin, or (4) pre-operative serum prostate-specific antigen (PSA) value >15 ng/ml, or a biopsy Gleason score >7, or a serum PSA level >10 ng/ml and a biopsy Gleason score >6. Based on data from D'Amico et al. (D'Amico et al., JAMA 284:1280-1283. 2000), these criteria were developed by the Southwest Oncology Group (protocol 9921) to identify men at intermediate to high risk of clinical recurrence after primary therapy. Disease status of the unaffected brothers was confirmed through serum PSA testing whenever possible.

The majority of the families were non-Hispanic white, although 13 African American and 2 Asian families were also included. The Institutional Review Board at the University of Michigan Medical School approved all aspects of the protocol, and all participants gave written informed consent, including permission to release their medical records.

### Genotyping Assays

Genomic DNA was isolated from whole blood using the Puregene kit (Gentra Systems Inc., Minneapolis, Minn.). A validated SNP, rs7223952, located 1,429 bases from the end of the BRCA1 transcript, was selected and genotyped from the Assays-On-Demand catalog (Applied Biosystems, Foster City, Calif.). After this SNP displayed significant association with prostate cancer, six additional SNPs were genotyped (rs2271539, rs691144, rs1799966, rs3737559, rs1799950, and rs799923) using the haplotype tagging strategy described below. All 7 SNPs were genotyped with the TaqMan allelic discrimination assay, and the ABI PRISM 7900HT Sequence Detection System (Applied Biosystems) to distinguish SNP alleles as previously described (Douglas et al., Cancer Epidemiol. Biomarkers Prev. 14:2035-2039. 2005). A genotyping call rate of 98.94%, with call rates >97.46% for each SNP was obtained. SNPs that were undetermined by the assay were sequenced for a final genotyping call rate of 100%. A subset of samples were also duplicated and verified by either TaqMan SNP genotyping or direct sequencing. Three discrepancies were observed among 363 duplicate genotype pairs by TaqMan SNP genotyping and 1 discrepancy among 166 duplicate genotype pairs by direct sequencing, yielding genotyping reproducibility rates of 99.2% and 99.4%, respectively.

### Haplotype Tagging Strategy

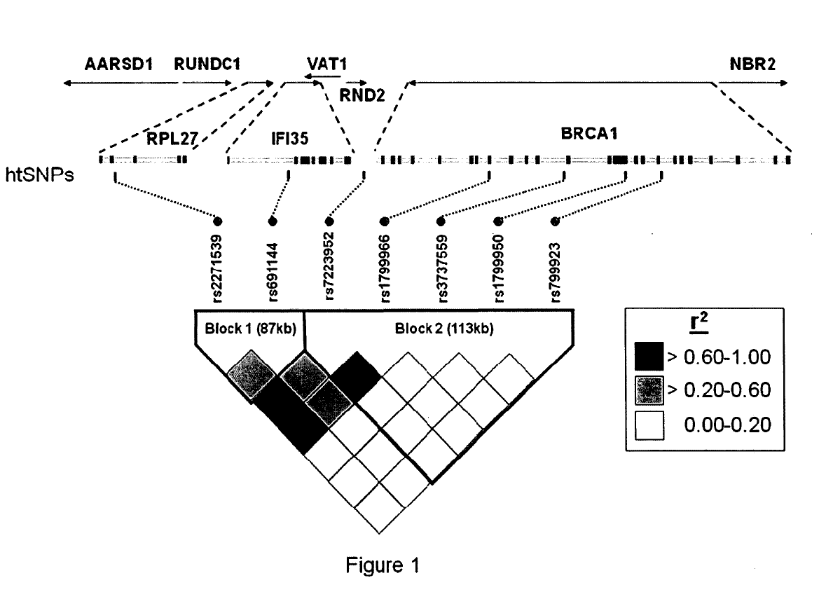

Based on the HapMap (Nature 437:1299-1320. 2005) data (February 2005 release), a total of 71 SNPs mapped to the 200 kb interval around the originally associated SNP, rs7223952, and 41 of these SNPs were present in at least one of the 60 CEU founders, i.e., unrelated individuals from a U.S. Utah population with northern and western European ancestry. Using these data, the dynamic programming algorithm proposed by Zhang et al. (Zhang et al., Proc. Natl. Acad. Sci. U.S.A 99:7335-7339. 2002) and implemented in HapBlock (Zhang et al., Bioinformatics. 21:131-134. 2005) (version 3.0) was used to partition this 200 kb region into blocks and select a maximally informative set of SNPs. Specifically, common haplotypes were defined as those having frequency >3% (i.e., haplotypes inferred to be present at least 4 times among 120 chromosomes), and a consecutive set of SNPs was defined as a block if common haplotypes accounted for at least 80% of all predicted haplotypes. Haplotype-tag SNPs (htSNPs) were then determined as the minimum set of SNPs that distinguished all common haplotypes inferred within each block. Based on these criteria, the 41 SNPs clustered into two, non-overlapping blocks of limited haplotype diversity, and six htSNPs (rs2271539 and rs691144 in the first block and rs1799966, rs3737559, rs1799950, and rs799923 in the second block) distinguished 94% and 96% of all haplotypes inferred within the first and second blocks, respectively (FIG. 1). SNP rs7223952, which is not present in the HapMap database, is located in the second block.

### Data Analysis Methods

The observed genotype distributions were tested for departures from Hardy-Weinberg equilibrium in a subset of unaffected, unrelated men by selecting the oldest unaffected man from each family. Two-SNP haplotype frequencies were estimated using the expectation-maximization algorithm and were used to calculate the LD measure R² between each pair of SNPs. Each of these analyses was conducted separately in two samples: non-Hispanic white men and African American men. For sake of comparison, minor allele frequencies, two-SNP haplotypes, and linkage disequilibrium were determined in unrelated individuals from the HapMap CEU and YRI samples (n=60 each), where YRI represents unrelated Yoruba people from Ibadan, Nigeria.

Conditional logistic regression with family was used as the stratification variable and a robust variance estimate that incorporates familial correlations due to potential linkage (Siegmund et al., Am. J. Hum. Genet. 67:244-248. 2000) to estimate odds ratios (OR's) and 95% confidence intervals (CI's) for the association between genotypes and prostate cancer. In parallel, the Family-Based Association Test (FBAT) package (version 1.5.5) was used to test for association between genotypes and prostate cancer. FBATs are a class of generalized score statistics that utilize within- and between-family marker-inheritance patterns to test for association (Laird et al., Genet. Epidemiol. 19 Suppl 1:S36-S42. 2000; Rabinowitz and Laird, Hum. Hered. 50:211-223. 2000) and, by design, eliminate any potential bias arising from population substructure. The empirical variance function in FBAT, which is a valid test of the null hypothesis of no association in the presence of linkage, was utilized. To maximize power, the combined sample of affected and unaffected men was analyzed using the offset option, which optimally weights the contribution of affected and unaffected subjects. Affecteds-only analyses was performed to allow for the possibility of misclassification of unaffected men (e.g., via reduced penetrance) and the resulting reduction in power. Both conditional logistic regression analyses and FBATs were carried out assuming additive, dominant, and recessive genetic models. For conditional logistic regression and affecteds-only FBATs, a general genotype model (i.e., 2 degrees of freedom model) was also tested. Predetermined stratified analyses were also performed to explore the relationship between genotypes and prostate cancer, stratifying on clinically advanced prostate cancer, age at diagnosis (<50 years), or number of confirmed cases of prostate cancer within a family (≧3).

To account for the number of correlated tests performed (i.e., 3 genetic models for each of 7 SNPs), permutation tests were performed to assess the overall significance of the primary FBAT results in the combined sample of affected and unaffected men. Specifically, 1,000 permuted samples were generated by randomly permuting the affection status labels of genotyped men within each sibship (i.e., leaving intact the vector of correlated SNPs). For each permuted sample, the FBATs described above were performed assuming additive, dominant, and recessive genetic models. The number of significant associations in the permuted samples was compared with the number of significant associations originally found. The overall p-value was computed as the proportion of permuted samples having at least as many tests with p-values less than or equal to the least significant p-value from the analyses of the original data.

To assess the association of haplotypes with prostate cancer, each of the htSNPs was divided by block and two- and four-SNP haplotypes corresponding to the first and second blocks, respectively were analyzed. Six-SNP haplotypes were also examined by combining SNPs from both blocks. Because the originally associated SNP, rs7223952, was in strong LD (R²>0.90) with one of the htSNPs, rs1799966, it was excluded from the haplotype-based analyses. All haplotypes were analyzed using the haplotype FBAT (HBAT) method (Horvath et al., Genet. Epidemiol. 26:61-69. 2004). All n-SNP haplotypes (where n=2, 4, or 6) were jointly tested for association with prostate cancer (i.e., a global test). Each individual haplotype was also tested for association with prostate cancer, assuming additive, dominant, and recessive genetic models. As described above for FBAT, the empirical variance option was used to account for prostate cancer linkage to this region and the offset option to weight the contribution of unaffected and affected subjects.

To determine whether the most strongly associated SNP, rs1799950, explained the previous evidence of prostate cancer linkage on 17q, it was genotyped in 172 of the original 175 GWS families (Lange et al., Prostate 57:326-334. 2003). These 172 families included 454 affected men. Of these 454 affected men, 129 from 76 of the 172 GWS families overlapped with the family-based association study and were already genotyped for rs1799950. Two different statistical methods were used to determine if the linkage signal was explained by rs1799950. First, the Genotype-IBD Sharing Test (GIST) proposed by Li et al (Am. J Hum. Genet. 74:418-431. 2004) was implemented in version 0.3 to investigate the correlation between genotypes at rs1799950 and family-specific non-parametric linkage (NPL) scores. The GIST assigns family-specific weights based on genotypes of affected family members and the model of interest (e.g., dominant) and tests for a positive correlation between this weight and family-based identity-by-descent (IBD) sharing as represented by the NPL score. Based on the 15 microsatellite markers on chromosome 17 from the original GWS (Lange et al., 2003, supra), non-parametric multipoint LOD (NPL) scores were recalculated using Merlin (Abecasis et al., Nat. Genet. 30:97-101. 2002) (version 1.0.0) with the ‘pairs’ scoring statistic, the exponential model, and equal weights for each of the 172 families. Second, the parametric likelihood framework recently developed by Li et al. (Am. J Hum. Genet. 76:934-949. 2005) was implemented in version 0.0.2 of the program to determine whether rs1799950 could fully or partly explain the original linkage signal on 17q. This method jointly models linkage and association (using genotypes at SNP rs1799950 and the 15 microsatellite markers on chromosome 17) to test for both linkage equilibrium and complete LD between the candidate SNP (i.e., rs1799950) and the putative prostate cancer susceptibility locus. Finally, to investigate the evidence for linkage in the families without the risk allele, we NPL scores were recalculated in the subset of families in which no affected men carried the risk allele.

All statistical tests were two-sided, and p-values <0.05 were considered statistically significant. Conditional logistic regression was conducted using version 8.2 of the SAS programming language (SAS Institute, Cary, N.C.). All remaining analyses (except where noted above) were conducted using the R language (version 2.1.1).

### B. Results

### Characteristics of the Families And Men

For this investigation, 338 families were identified with at least one DSP, resulting in a total of 546 DSPs. Of the 338 families, 331 included only the index case and one or more of his brothers. The remaining 7 families included additional DSPs unrelated to the index case as a brother (e.g., a pair of DSPs related as first cousins). Of the 338 families, ˜32%, 37%, and 31% included one, two, and three or more men with prostate cancer, respectively. In total, the entire sample consisted of 860 men (459 affected and 401 unaffected men). The majority of men were non-Hispanic white (434 affected and 383 unaffected men). The clinical characteristics of the men with prostate cancer are shown in Table 1. The median age at diagnosis was 55 years (inter-quartile range=50-63 years). The median age of unaffected men at their time of consent was 56 years (inter-quartile range=50-63 years). Over 75% of unaffected men reported their most recent PSA testing results and/or had their PSA values confirmed by medical record review, and ˜94% of them reported and/or had a prostate-specific antigen level <4.0 mg/dL or normal. At the time of consent, unaffected men were significantly older than their affected brothers were at their time of diagnosis (p<0.0001 for paired t-test of within family means), with a mean age difference of ˜3 years.

### Allele Frequencies And Disequilibrium Analyses

Initially, SNP rs7223952, which is located ˜1.4 kb downstream from the transcription stop site of the BRCA1 gene on chromsome 17q, was genotyped. Based on the haplotyping tagging strategy described above (see Materials and Methods), six additional SNPs spanning a 200 kb region around rs7223952 were genotyped. These SNPs include the following: rs2271539 in intron 2 of the RPL27 gene, rs691144 in intron 1 of the IF135 gene, and rs1799966, rs3737559, rs1799950, and rs799923 in exon 16, intron 13, exon 11, and intron 6, respectively, of the BRCA1 gene. Positions and minor allele frequencies for all seven SNPs are given in Table 2 for four different samples, including the two ethnic-specific samples of unrelated, unaffected men and HapMap CEU and YRI samples. Allele frequencies for these SNPs varied considerably among samples. Two of the SNPs, rs1799950 and rs799923, were monomorphic in the sample of unrelated, unaffected African American men and the sample of unrelated Yoruba people from the HapMap project. The observed genotype data were consistent with Hardy-Weinberg equilibrium in the two ethnic-specific samples of unrelated, unaffected men.

The pattern of LD in the 200-kb region surrounding rs7223952 is shown in FIG. 1 for the sample of unrelated, unaffected men of non-Hispanic white descent. Consistent with our haplotyping tagging strategy, 16 of all 21 SNP pairs exhibited weak LD (R²<0.2). SNP rs7223952, which was not present in the HapMap database and therefore did not inform the htSNP selection strategy, was in strong LD with htSNP rs1799966 (R²=0.94). In general, the LD patterns in FIG. 1 were consistent with those predicted by the CEU HapMap sample. Pair-wise LD (as measured by R²) also differed substantially between the samples of non-Hispanic white and African American men and between the HapMap CEU and YRI samples. The results below are based on the 323 families of non-Hispanic white descent, i.e., after excluding 15 of 338 families (<5%).

### Single SNP And Haplotype-Based Association Analyses

Table 3 summarizes results for all seven SNPs using both conditional logistic regression and family-based association tests. Before conducting the conditional logistic regression analysis, men were excluded who were not brothers of the index case. The resulting sample consisted of 799 men and 506 DSPs. Initial FBAT analyses included only affected men (n=434), while the primary, combined analyses (reported below) included all affected and unaffected men (n=817), resulting in a total of 516 DSPs. An association between prostate cancer and SNP rs7223952 downstream of the BRCA1 gene was identified. After genotyping an additional 6 htSNPs, the strongest evidence for prostate cancer association was for SNP rs1799950, which results in a glutamine-to-arginine substitution at codon 356 (Gln356Arg) in exon 11 of the BRCA1 gene. Under a dominant model for disease (i.e., presence of one or both copies of the minor allele versus no copies of the minor allele, where the latter is the referent group), the minor allele of rs1799950 was preferentially transmitted to affected men (z=2.79; p=0.005), with an odds ratio of 2.25 (95% CI=1.21-4.20; p-value=0.011). Another SNP in the BRCA1 gene, rs3737559 in intron 13, also revealed significant evidence of prostate cancer association under a dominant model (z=−2.37; p=0.018). Both SNPs were less strongly but still significantly associated with prostate cancer under an additive model. Under an additive (but not dominant) genetic model, the minor allele at SNP rs799923 in intron 6 of the BRCA1 gene was preferentially transmitted to affected men (z=2.07; p=0.039) (data not shown). Each of these associated SNPs (i.e., rs1799950, rs3737559, and rs799923) were in weak LD with each other and with rs7223952 in our sample of unrelated, unaffected men (R²<0.2) (FIG. 1). No evidence was found of an association between SNPs rs2271539, rs691144, or rs1799966 and prostate cancer. Thus, a total of 3 SNPs exhibited significant prostate cancer association in 5 FBATs for the combined sample of affected and unaffected men. Among 1,000 permuted samples, only 24 samples had 5 or more FBAT results with a p-value ≦0.039, indicating that these association results are unlikely to be due to chance alone (p=0.024).

As described above, analyses were repeated after stratifying on clinically aggressive prostate cancer, age at diagnosis (<50 years), and number of confirmed cases of prostate cancer within a family (≧3). The following results were based on a dominant model but were also statistically significant under an additive model. After stratification, the minor allele at SNP rs1799950 was preferentially transmitted to affected men in the subset of families in which affected men were diagnosed with prostate cancer at <50 years of age (z=2.89; p=0.004), with an odds ratio of 7.51 (95% CI=0.74-76.23; p=0.088). In the subset of families with 3 or more confirmed affected men, the minor allele at SNP rs3737559 was preferentially transmitted to unaffected men (z=−2.49; p=0.013). These stratified results are consistent with the FBAT results based on all families and are statistically significant despite a substantial loss of informative families, over half in both cases (12 versus 49 families for rs1799950 and 20 versus 47 families for rs3737559). No significant evidence of an association between SNPs rs691144, rs7223952, rs1799966, or rs799923 and prostate cancer was found in any of the stratified analyses. Although SNP rs2271539 was not significantly associated with prostate cancer in the un-stratified analyses, the major allele was preferentially transmitted to affected men in the subset of families with 3 or more confirmed affected men (z=−2.53; p=0.012), with an odds ratio of 0.40 (95% CI=0.17-0.95; p=0.038).

Haplotype analysis did not reveal a risk haplotype or set of haplotypes that explained the prostate cancer associations substantially more than individual SNPs. Further, the only significantly associated haplotype contained the most significantly associated SNP, rs1799950. For example, the 4-SNP haplotype uniquely defined by the minor allele at rs1799950 was over-transmitted to affected men under both dominant (z=2.812; p=0.005) and additive genetic models (z=2.589; p=0.01), consistent with single-SNP results for rs1799950. Similarly, the 6-SNP haplotype uniquely defined by the minor allele of rs1799950 was also significantly over-transmitted to affected men under both dominant and additive models.

### Confirmatory Linkage Analyses

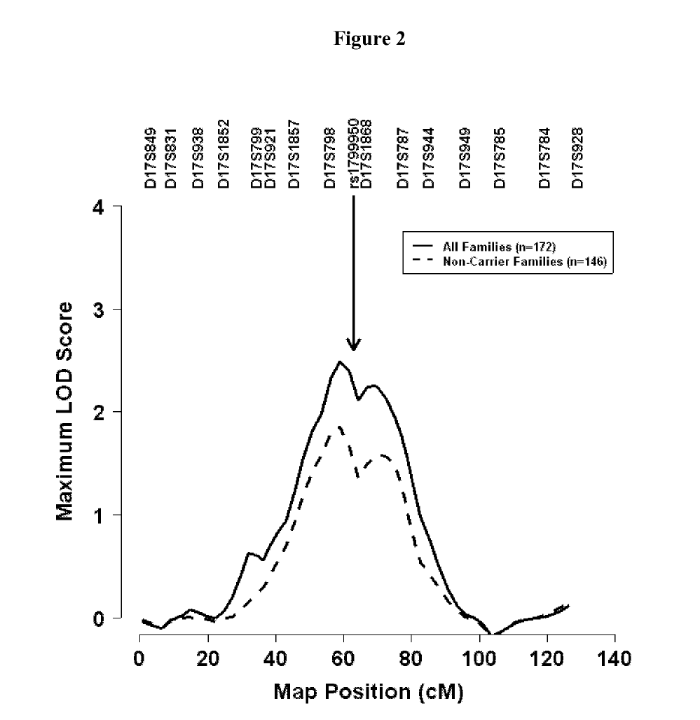

The most significant SNP, rs1799950, was followed up by genotyping it in the GWS families and using the GIST to test whether it explained the original linkage signal on 17q. At least one affected man carrying at least one copy of the minor allele at rs1799950 was present in 26 of the 172 GWS families. Under the dominant model suggested by the association analysis, evidence was found for an association between the presence of the risk (or minor) allele in affected men and increased IBD allele sharing among brothers affected with prostate cancer (p=0.035), suggesting that SNP rs1799950 may partially explain the originally reported linkage signal on 17q (Lange et al., 2003, supra). Based on a parametric method, evidence of linkage (p=0.02) was reconfirmed but the null hypothesis that SNP rs1799950 is in complete LD with the putative prostate cancer susceptibility locus (p=0.008) was rejected, suggesting that rs1799950 does not fully account for the linkage signal on 17q. Consistent with these findings, evidence of suggestive prostate cancer linkage to 17q21 remained after the removal of families in which affected men carried one or more copies of the minor allele at rs1799950 (MLS of 1.85 in 146 non-carrier families versus 2.49 in all 172 families) (FIG. 2).

TABLE 1
Characteristics of men with prostate cancer (n = 459)

| Trait | No.* (%) |
|---|---|
| Age at diagnosis† | 55 [50-63] |
| Pre-diagnosis PSA† | 5.9 [4.3-10.0] |
| Surgery‡ (% yes) | 349 (76%) |

Stage:

| | |
|---|---|
| Localized | 338 (77%) |
| Locally advanced | 84 (19%) |
| Metastasized | 17 (4%) |

Gleason:

| | |
|---|---|
| ≦6 | 220 (49%) |
| 7 | 180 (40%) |
| >7 | 48 (11%) |
| Clinically aggressive CaP (%) | 167 (36%) |

*Note that column subtotals do not sum to 459 due to missing data.
†Median and [interquartile range] are reported.
‡Number and (percentage) of men with prostate cancer who underwent a radical prostatectomy.

TABLE 2
Minor allele frequencies for SNPs in or near the BRCA1 gene

| | | | | | DSP* | | | |
|---|---|---|---|---|---|---|---|---|
| | | | | Major > | Non-Hispanic | African | HapMap† | |
| | | | | minor | whites | Americans | CEU | YRI |
| Gene | SNP name | Location | Position‡ | allele | (n = 323) | (n = 13) | (n = 60) | (n = 60) |
| RPL27 | rs2271539 | Intron 2 | −43.936 | T > C | 0.30 | 0.69 | 0.36 | 0.85 |
| IFI35 | rs691144 | Intron 1 | −31.236 | G > A | 0.19 | 0.23 | 0.23 | — |
| Intergenic | rs7223952 | — | 0 | A > G | 0.31 | 0.92 | — | — |
| BRCA1 | rs1799966 | Exon 16 | 28.209 | T > C | 0.30 | 0.27 | 0.35 | 0.21 |
| BRCA1 | rs3737559 | Intron 13 | 39.419 | G > A | 0.08 | 0.04 | 0.07 | 0.01 |
| BRCA1 | rs1799950 | Exon 11 | 51.596 | A > G | 0.07 | 0 | 0.04 | 0 |
| BRCA1 | rs799923 | Intron 6 | 57.046 | G > A | 0.25 | 0 | 0.31 | 0 |

*Data are from the DSP (discordant sib pair) sample: oldest, unaffected man from each family.
†Data are from HapMap samples: unrelated Utah residents with ancestry from northern and western Europe (CEU) and unrelated Yoruba people ascertained from Ibadan, Nigeria (YRI).
‡Kilobase position relative to SNP rs7223952 according to the reference sequence (UCSC Genome Browser, NCBI Build 36.1, March 2006).
Note:
Entries marked by a dash (—) indicate that the corresponding SNP was not in the HapMap database.

TABLE 3
Association results from conditional logistic regression and family-based association tests

| | | | | | | | | Affected and | | |
|---|---|---|---|---|---|---|---|---|---|---|
| | | Discordant sib pairs | | | Affected men | | | unaffected men | | |
| | | Odds | | P- | | Z- | P- | | Z- | P- |
| SNP name | Genotype* | ratio | 95% CI | value | Families† | score | value | Families† | score | value |
| rs2271539 | CC vs CT | 0.69 | (0.47, 1.01) | 0.06 | 117 | −1.73 | 0.08 | 119 | −1.78 | 0.08 |
| | or TT | | | | | | | | | |
| rs691144 | GG vs GA | 0.82 | (0.54, 1.24) | 0.34 | 97 | −0.91 | 0.36 | 100 | −0.93 | 0.35 |
| | or AA | | | | | | | | | |
| rs7223952 | AA vs AG | 0.70 | (0.47, 1.04) | 0.08 | 112 | −1.89 | 0.06 | 113 | −1.84 | 0.07 |
| | or GG | | | | | | | | | |
| rs1799966 | TT vs TC | 0.70 | (0.47, 1.05) | 0.08 | 114 | −1.83 | 0.07 | 115 | −1.81 | 0.07 |
| | or CC | | | | | | | | | |
| rs3737559 | GG vs GA | 0.43 | (0.21, 0.89) | 0.02 | 45 | −2.20 | 0.03 | 47 | −2.37 | 0.02 |
| | or AA | | | | | | | | | |
| rs1799950 | AA vs AG | 2.25 | (1.21, 4.20) | 0.01 | 49 | 2.66 | <0.01 | 49 | 2.79 | <0.01 |
| | or GG | | | | | | | | | |
| rs799923 | GG vs GA | 1.46 | (0.98, 2.17) | 0.06 | 106 | 1.90 | 0.06 | 106 | 1.89 | 0.06 |
| | or AA | | | | | | | | | |

*First genotype is the referent group.
†Number of informative families.
Note:
Data are based on 323 families of non-Hispanic white descent.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of diagnosing prostate cancer, comprising
a) detecting the presence or absence of a BRCA1 single nucleotide polymorphism, wherein said polymorphism is rs1799950 in a sample from a subject; and
b) determining the presence or absence of prostate cancer in said sample based on said presence or absence of said BRCA1 single nucleotide polymorphism.

2. The method of claim 1, wherein the presence of said BRCA1 single nucleotide polymorphism in said sample is indicative of prostate cancer in said sample.

3. The method of claim 1, wherein said rs1799950 single nucleotide polymorphism corresponds to a Gln356Arg substitution in a BRCA1 polypeptide expressed from said rs1799950 single nucleotide polymorphism in BRCA1.

4. The method of claim 1, wherein said sample is a biopsy sample.

5. The method of claim 1, wherein said sample is urine.

6. The method of claim 1, wherein said sample is blood.

7. A method of diagnosing prostate cancer, comprising
a) detecting the presence or absence of a BRCA1 single nucleotide polymorphism, wherein said polymorphism is rs3737559 in a sample from a subject; and
b) determining the presence or absence of prostate cancer in said sample based on said presence or absence of said BRCA1 single nucleotide polymorphism.

8. The method of claim 7, wherein the presence of said BRCA1 single nucleotide polymorphism in said sample is indicative of prostate cancer in said sample.

9. A method of diagnosing prostate cancer, comprising
a) detecting the presence or absence of a BRCA1 single nucleotide polymorphism, wherein said polymorphism is rs799923 in a sample from a subject; and
b) determining the presence or absence of prostate cancer in said sample based on said presence or absence of said BRCA1 single nucleotide polymorphism.

10. The method of claim 9, wherein the presence of said BRCA1 single nucleotide polymorphism in said sample is indicative of prostate cancer in said sample.

11. A kit comprising reagents for detecting the presence or absence of a BRCA1 single nucleotide polymorphism selected from the group consisting of rs1799950, rs3737559, and rs799923.

12. A method, comprising:
a) contacting a prostate cancer cell expressing a BRCA1 gene comprising a BRCA1 single nucleotide polymorphism selected from the group consisting of rs1799950, rs3737559, and rs799923 with a test compound;
b) identifying a test compound that impairs proliferation of or kills said cell.

13. The method of claim 12, further comprising the step of administering said test compound that impairs proliferation of or kills said cell to a subject diagnosed with prostate cancer.

14. The method of claim 12, further comprising the step of marketing said test compound as a drug for treating prostate cancer.
